(19) United States
(12) Patent Application Publication
(10) Pub. No.: US 2024/0245887 A1
(43) Pub. Date: Jul. 25, 2024

(54) LIGHT-EMITTING DIODE CATHETER

(71) Applicant: Crossfire Medical, Inc., Dover, DE (US)

(72) Inventors: Adam Tschida, Brooklyn Park, MN (US); Ryan Egeland, Plymouth, MN (US)

(21) Appl. No.: 18/417,134

(22) Filed: Jan. 19, 2024

Related U.S. Application Data
(60) Provisional application No. 63/481,386, filed on Jan. 24, 2023.

(51) Int. Cl. A61M 25/01

(52) U.S. Cl. CPC A61M 25/0105; A61M 2205/587

(57) ABSTRACT

The present disclosure includes a method of locating a treatment site of a patient and treating a vascular disease, comprising inserting a wire into a vessel of the patient. In some examples, the wire has a distal end and a proximal end located opposite the distal end and is coupled to a control device at the proximal end. The method may comprise an at least one light- emitting diode (LED) disposed in a distal portion of the wire, the distal portion is adjacent the distal end. According to some examples, the method comprises illuminating, via the control device, the at least one LED within the vessel of the patient. The method may comprise locating, extracorporeally, the at least one LED. In some examples, the method includes positioning the wire via the at least one LED so that the distal end is located adjacent to the treatment site.

### CROSS-REFERENCE TO RELATED APPLICATIONS

The entire contents of the following application are incorporated by reference herein: U.S. Provisional Patent Application No. 63/481,386; filed Jan. 24, 2023; and entitled LIGHT-EMITTING DIODE CATHETER.

### INTRODUCTION

### Technical Field

The present disclosure relates to locating a catheter. Specifically, the present disclosure relates to locating a catheter within a patient's body.

### Background

Many intravascular procedures include some means of locating a catheter and wire within the body of a patient. It is desirable to locate these objects to prevent damage to unintended vessel walls and ensure treatment is provided in the correct location. Current solutions to this problem rely on using echogenic features to locate intravenous catheters and wires or on cameras whose view can penetrate a patient's body. However, these solutions in the prior art have several shortcomings. The systems and methods disclosed herein seek to remedy these deficiencies in the prior art.

### SUMMARY

Included in the present disclosure is a method of locating a treatment site of a patient. In some examples, the method of locating a treatment site of a patient includes inserting a wire into a vessel of the patient (e.g., step 600 as shown in FIG. 6). According to some examples, the wire includes a distal end and a proximal end located opposite the distal end. The wire may be coupled to a control device at the proximal end. In some examples, at least one light-emitting diode (LED) is disposed in a distal portion of the wire, the distal portion adjacent the distal end. According to some examples, the method of locating a treatment site of a patient includes illuminating, via the control device, the at least one LED within the vessel of the patient (e.g., step 602 as shown in FIG. 6). The method of locating a treatment site of a patient may include locating, extracorporeally, the at least one LED (e.g., step 604 as shown in FIG. 6). In some examples, the method of locating a treatment site of a patient includes positioning, via the at least one LED, the wire such that the distal end is located adjacent to the treatment site (e.g., step 606, as shown in FIG. 6).

Also included in the present disclosure is a method of locating a treatment site of a patient. In some examples, the method of locating a treatment site of a patient includes inserting a catheter into a vessel of a patient (e.g., step 900 as shown in FIG. 9). According to some examples, the catheter includes a sheath having a working lumen, a proximal sheath end, and a distal sheath end opposite the proximal sheath end. The proximal sheath end may be coupled to a control device. In some examples, the distal sheath end is configured for insertion into a vessel of a patient. According to some examples, the catheter includes a wire extending from the control device through the working lumen of the sheath to the distal sheath end. The wire may include a proximal wire end and a distal wire end opposite the proximal wire end. In some examples, at least one light-emitting diode (LED) is disposed in the distal wire end. According to some examples, the method of locating a treatment site of a patient includes illuminating, via the control device, the at least one LED within the vessel of the patient (e.g., step 902 as shown in FIG. 9). The method of locating a treatment site of a patient may include locating, extracorporeally, the at least one LED (e.g., step 904 as shown in FIG. 9), and positioning, via the at least one LED, the catheter such that the distal wire end is located adjacent to the treatment site (e.g., step 906 as shown in FIG. 9). In some examples, the method of locating a treatment site of a patient includes retracting the sheath to expose the distal wire end from the distal sheath end (e.g., step 908 as shown in FIG. 9).

Also included in the present disclosure is a medical device (e.g., the medical device 10 as shown in FIG. 1) for locating a treatment site (e.g., the treatment site 50 as shown in FIG. 1). In some examples, the medical device for locating a treatment site includes a wire (e.g., the wire 30 as shown in FIG. 1) having a distal wire end (e.g., the distal wire end 308 as shown in FIG. 3A) and a proximal wire end (e.g., the proximal wire end 306 as shown in FIG. 3A) located opposite the distal wire end. According to some examples, the wire is coupled to a control device (e.g., the control device 20 as shown in FIG. 1) at the proximal wire end. The medical device for locating a treatment site may include at least one light-emitting diode (LED) (e.g., the at least one LED 302a, 302b, and 302c as shown in FIG. 3A) disposed in a distal portion (e.g., the distal portion 310 as shown in FIG. 3B) of the wire, the distal portion adjacent to the distal wire end. In some examples, the at least one LED is configured to radially illuminate a treatment site such that the treatment site is visible extracorporeally. According to some examples, the control device is configured to provide power to the at least one LED.

The foregoing, and other features and advantages of the invention, will be apparent from the following, more particular description of the preferred embodiments of the invention, the accompanying drawings, and the claims.

### BRIEF DESCRIPTION OF THE DRAWINGS

These and other features, aspects, and advantages are described below with reference to the drawings, which are intended to illustrate, but not to limit, the invention. In the drawings, like characters denote corresponding features consistently throughout similar embodiments.

FIG. 1 illustrates a diagrammatic view of a medical device as it may appear inserted into a patient's vasculature, according to some examples.

FIG. 2 illustrates a diagrammatic view of a medical device as it may appear when located adjacent to a treatment site, according to some examples.

FIG. 3A illustrates a profile view of a catheter wire including light-emitting diodes (LEDs) throughout, according to some examples.

FIG. 3B illustrates a profile view of a catheter wire including at least a distal LED, according to some examples.

FIG. 3C illustrates a profile view of a catheter wire including combinations of LEDs, as presented in FIGS. 3A and 3B, according to some examples.

FIG. 4A illustrates a profile view of a catheter wire including LEDs throughout, according to some examples.

FIG. 4B illustrates a profile view of a catheter wire including at least a distal LED, according to some examples.

FIG. 4C illustrates a profile view of a catheter wire including combinations of LEDs, as presented in FIGS. 4A and 4B, according to some examples.

FIG. 5 illustrates a diagrammatic, side, cross-sectional view of a medical device, according to some examples.

FIG. 6 illustrates a flow chart depicting a method of locating a wire within a treatment site, according to some examples.

FIG. 7 illustrates a flow chart depicting a method of abrading a treatment site, according to some examples.

FIG. 8 illustrates a flow chart depicting a method of abrading an additional treatment site, according to some examples.

FIG. 9 illustrates a flow chart depicting a method of locating at least one LED, according to some examples.

FIG. 10 illustrates a flow chart depicting a method of abrading and injecting a fluid- type drug into a treatment site, according to some examples.

FIG. 11 illustrates a flow chart depicting a method of abrading an additional treatment site, according to some examples.

### COMPONENT INDEX

10—Medical device
20—Control device
30—Wire
40—Catheter
50—Treatment site
60—Patient
202—At least partially sinusoidal shape
204—Sheath
302—At least one light-emitting diode (LED)
302a—At least one LED
302b—At least one LED
302c—At least one LED
304—Working lumen
306—Proximal wire end
308—Distal wire end
310—Distal portion
312—Proximal sheath end
31—Distal sheath end
316—Distal LED
502—Motor
504—Actuator
600, 602, 604, and 606—Method steps
700, 702, 704, and 706—Method steps
800, 802, 804, and 806—Method steps
900, 902, 904, 906, and 908—Method steps
1000, 1002, 1004, 1006, and 1008—Method steps
1100, 1102, 1104, 1106, and 1108—Method steps

### DETAILED DESCRIPTION

The present disclosure describes systems and techniques for treating vascular disorders such as varicose veins. Some existing prior art systems include the use of highly complicated interventional devices (e.g., ablation catheters), which involve significant user training to enable correct and effective use due to the devices' requirements for the user to multitask while performing complicated dexterous techniques.

The present disclosure describes systems and methods for locating a catheter 40 and a wire within a patient's body. These techniques may include using light-emitting diodes (LEDs) at one or more positions along the catheter or wire. Some existing solutions for locating a catheter and wire in a patient's body include the use of echogenic components and ultrasound and radiographs. The present disclosure permits visual, extracorporeal confirmation of catheter and wire location. Additionally, the present disclosure may not require the use of additional imaging equipment, thus decreasing costs of both component manufacture and procedural costs.

The present disclosure may also permit the light from one or more LEDs to be visible from outside of the patient, as well as the wire to be located without the use of expensive and sometimes cost-prohibitive imaging devices, such as ultrasound or radiograph devices. The present disclosure may also permit the exact location of the catheter or wire to be confirmed in conjunction with other already-known methods.

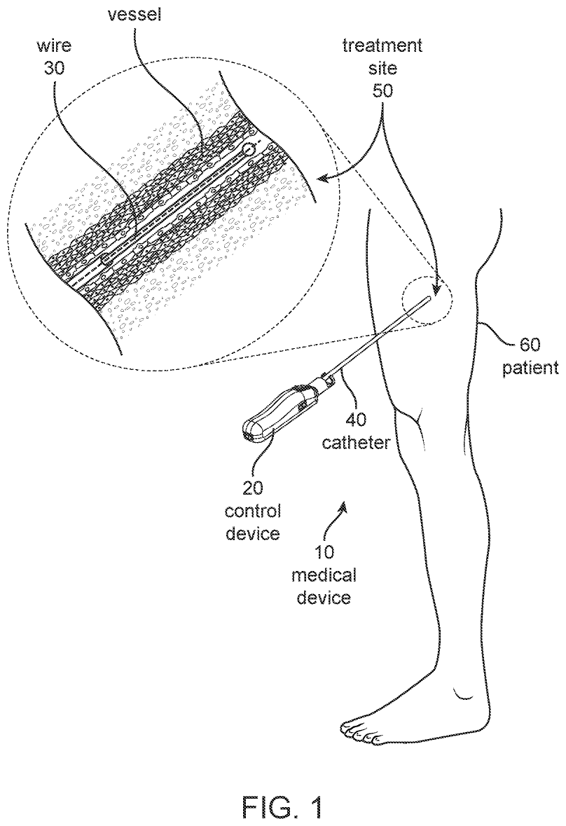

FIG. 1 illustrates one possible diagrammatic view of a medical device 10. The medical device 10 includes a control device 20 that may be removably coupled to a catheter 40. The catheter 40 may enter a patient 60 at a treatment site 50. While FIG. 1 shows the point of insertion located on a human leg, this is not intended to be limiting, and the treatment site 50 may be located anywhere on the patient's body 60 (i.e., the common femoral artery, the radial artery, etc.). It is also understood that the patient 60 is not required to be human for the medical device 10 to function. FIG. 1 also illustrates the control device 20 with a specific shape and configuration, although no specific shape of the control device 20 is required for the medical device 10 to function.

FIG. 1 also illustrates an inset view of a treatment site 50 in the patient 60, along with the surrounding vasculature. This inset view also shows the catheter 40 and a wire 30 extending into said vasculature. However, it is understood that the medical device 10 may function with either the wire 30 or the catheter 40 in isolation, when both the catheter 40 and the wire 30 are present, and when neither the catheter 40 nor the wire 30 are present. Additionally, neither the wire 30 nor the catheter 40 are required to be straight, as shown in FIG. 1. FIG. 1 also illustrates the wire 30 with a tip having a diameter different than the diameter of the body of the wire 30. However, this also is not required.

Throughout the specification, the catheter 40 is disclosed as comprising a wire 30. However, it is understood that the present specification is not limited to using a wire 30. The present specification also enables the use of a hypotube, a catheter shaft, combinations thereof, and combinations of a hypotube and/or catheter shaft with a wire 30.

Additionally, the use of the term “wire” is not intended to be limiting, and the present disclosure may be extended to or used in conjunction with any catheter, stent delivery system, endovascular catheter, endovascular wire, balloon catheter, atherectomy catheter, exchange catheter, guidewire catheter, crossing catheter, etc. Generally, any application that would benefit from visualizing the placement of a wire, or equivalent, through the skin, either subcutaneous or endovascular, may benefit from the present disclosure.

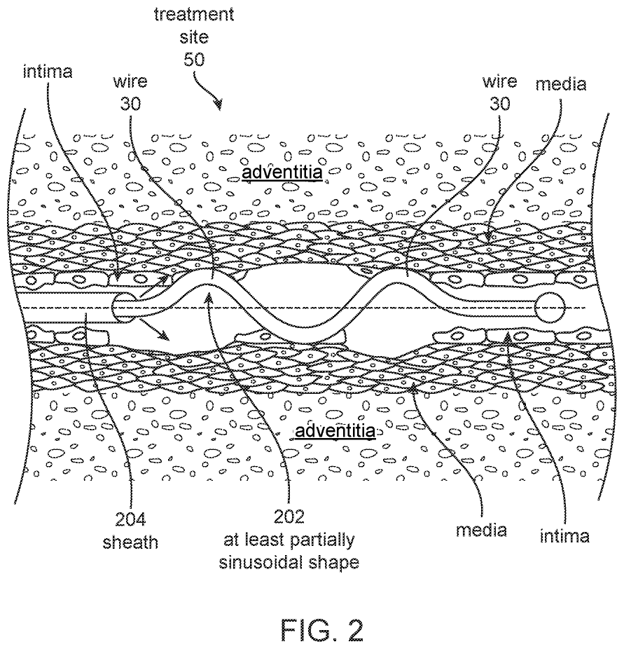

FIG. 2 illustrates a diagrammatic view showing how the medical device 10 may appear within a treatment site 50. The treatment site 50 may include the wire 30 extending through the vessel. The treatment site 50 may also include a sheath 204, which may at least partially surround the wire 30. The sheath 204 may be used to advance the wire 30 through the vessel of the patient 60. FIG. 2 also shows the wire 30 to have an at least partially sinusoidal shape 202. However, the wire 30 may be straight or have any profile between straight and sinusoidal, including two-dimensional profiles, three-dimensional profiles, and combinations thereof. Examples of two-dimensional profiles include but are not limited to shapes with points, such as triangles, shapes with curves, such as circles, and combinations thereof. The wire 30 may include a diameter that is consistent, varying, growing, decreasing, and combinations thereof throughout its length.

The partially sinusoidal shape 202 may allow the wire 30 to abrade the intima of the vessel, possibly destroying sections of the intima and abrading into the media of the vessel. This abrading may also be done by causing the wire 30 to turn, spin, move, or any combination of these. The vessel wall may be abraded for sclerotherapy purposes, although abrading may be done for any reason. FIG. 2 also illustrates the wire 30 to include a tip having a diameter different than the diameter of the body of the wire 30, although this is not required.

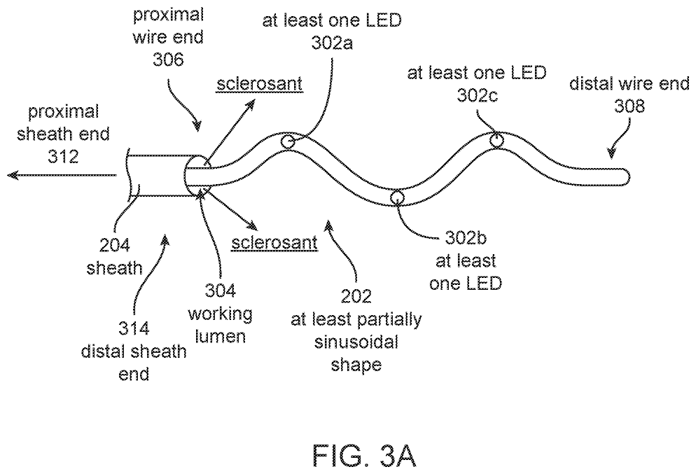

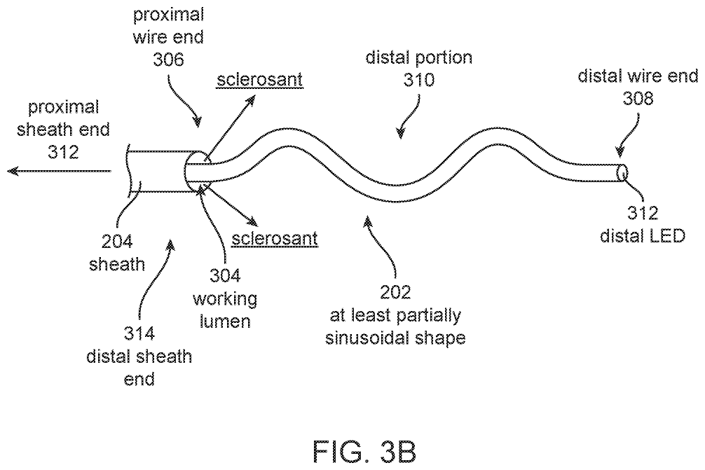

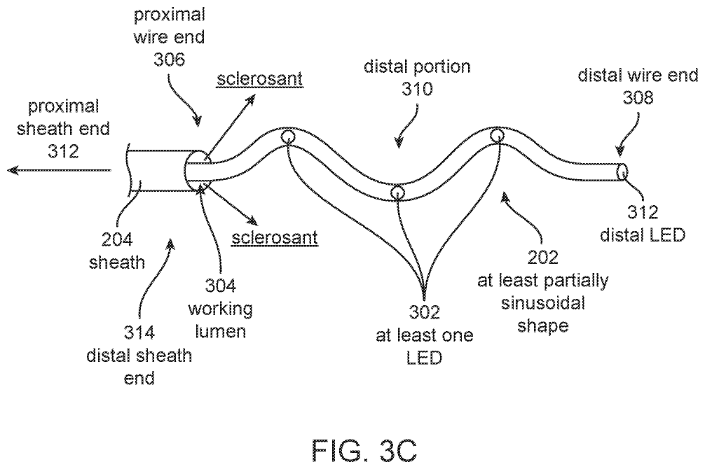

FIG. 3A illustrates a profile view of a catheter wire 30 including LEDs throughout. According to FIG. 3A, the catheter wire 30 includes a proximal wire end 306 and a distal wire end 308 opposite the proximal wire end 306. While the sheath 204 is not required, it is also shown in FIG. 3A, including a distal sheath end 314 and a proximal sheath end 312 opposite the distal sheath end 314.

Additionally, FIG. 3A illustrates a working lumen 304 with sclerosant being injected from the distal sheath end 314. The working lumen 304 is not required to inject sclerosant, nor is the working lumen 304 required to be within a sheath 204. The injection of sclerosant is in addition to the already mentioned abrading capabilities of FIG. 2, and it is understood that it is not necessary for the medical device 10 to inject sclerosant to be enabled, nor does the injection of sclerosant have any bearing on the extracorporeal visibility of the medical device 10.

FIG. 3A further illustrates the wire 30 to include the at least partially sinusoidal shape 202. However, the wire 30 may be straight or have any profile between straight and sinusoidal, including two-dimensional profiles, three-dimensional profiles, and combinations thereof. Examples of two-dimensional profiles include but are not limited to shapes with points, such as triangles, shapes with curves, such as circles, and combinations thereof. These shapes may allow the wire 30 to be physically visible extracorporeally. The wire 30 may include a diameter that is consistent, varying, growing, decreasing, and combinations thereof throughout its length.

It is also illustrated in FIG. 3A that the wire 30 includes three examples of locations for at least one LED 302a, 302b, and 302c; in this example, placed along the bends of the at least partially sinusoidal shape 202. While three LEDs (302a, 302b, and 302c) are illustrated in FIG. 3A, this is not intended to be limiting, and it is understood that as few as one LED 302 and as many LEDs 302 as may fit within the body of the wire 30 may be located within the wire 30 and at any place along the wire 30.

The wire 30 is disclosed throughout the specification as possibly comprising at least one LED 302. However, it is understood that the present specification is not solely limited to LEDs 302 located within a wire 30. The present specification also enables the use of a hypotube, a catheter shaft, combinations thereof, and in combination with a wire 30 for the location of the at least one LED 302. Additionally, the at least one LED 302 may be in or on the surface of any of these components.

FIG. 3B illustrates a profile view of a catheter wire 30, including at least a distal LED 316. The catheter wire 30 includes a proximal wire end 306 and a distal wire end 308 opposite the proximal wire end 306. While the sheath 204 is not required, FIG. 3B also shows the sheath 204 with a distal sheath end 314 and a proximal sheath end 312 opposite the distal sheath end 314. FIG. 3B additionally illustrates a working lumen 304 with sclerosant being injected from the distal sheath end 314. The working lumen 304 is not required to inject sclerosant, nor is the working lumen 304 required to be within a sheath 204. The injection of sclerosant is in addition to the already mentioned abrading capabilities of FIG. 2, and it is understood that it is not necessary for the medical device 10 to inject sclerosant to be enabled, nor does the injection of sclerosant have any bearing on the extracorporeal visibility of the medical device 10.

FIG. 3B further illustrates that the catheter wire 30 may include a distal portion 310 of the wire 30, including a distal LED 316 at the distal wire end 308. This figure also shows no other LEDs 302 located in the distal portion 310. However, the catheter wire 30 may include one or more LEDs 302 and may be located at any place along the wire 30, including the distal portion 310. A single distal LED 316 may be beneficial in some instances, such as when the termination point of the wire 30 at the distal tip is deemed important to be located with greater accuracy. However, multiple LEDs 302 along the length of the wire 30 may also be beneficial in some instances, such as when the entirety of a section of the wire 30 is deemed important to be located with greater accuracy. However, neither format of LEDs 302 is hereby deemed more beneficial than the other in every instance, and they may be used separately, with each other, with other formats, or combinations thereof.

FIG. 3C illustrates a profile view of a catheter wire 30 including combinations of LEDs 302 as presented in FIGS. 3A and 3B. The catheter wire 30 includes a proximal wire end 306 and a distal wire end 308 opposite the proximal wire end 306. While the sheath 204 is not required, FIG. 3C also shows the sheath 204 with a distal sheath end 314 and a proximal sheath end 312 opposite the distal sheath end 314. FIG. 3C additionally illustrates a working lumen 304 with sclerosant being injected from the distal sheath end 314. The working lumen 304 is not required to inject sclerosant, nor is the working lumen 304 required to be within a sheath 204. The injection of sclerosant is in addition to the already mentioned abrading capabilities of FIG. 2, and it is understood that it is not necessary for the medical device 10 to inject sclerosant to be enabled, nor does the injection of sclerosant have any bearing on the extracorporeal visibility of the medical device 10.

FIG. 3C further illustrates the wire 30 to include the at least partially sinusoidal shape 202. However, the wire 30 may be straight or have any profile between straight and sinusoidal, including two-dimensional profiles, three-dimensional profiles, and combinations thereof. Examples of two-dimensional profiles include but are not limited to shapes with points, such as triangles, shapes with curves, such as circles, and combinations thereof. These shapes may allow the wire 30 to be physically visible extracorporeally. The wire 30 may include a diameter that is consistent, varying, growing, decreasing, and combinations thereof throughout its length. It is also illustrated in FIG. 3C that the wire 30 includes three examples of locations for the at least one LED 302, in this example placed on the bends in the at least partially sinusoidal shape 202 and all in the distal portion 310, as well as the distal LED 316. However, the catheter wire 30 may include one or more LEDs 302 and may be located at any place along the wire 30.

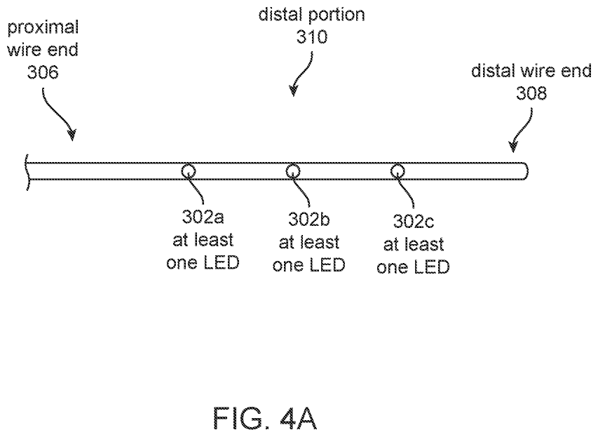

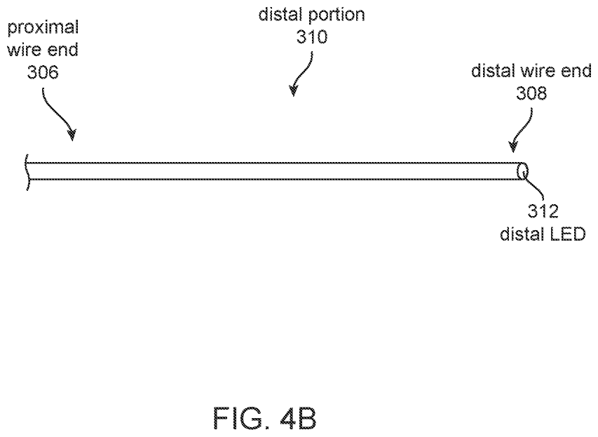

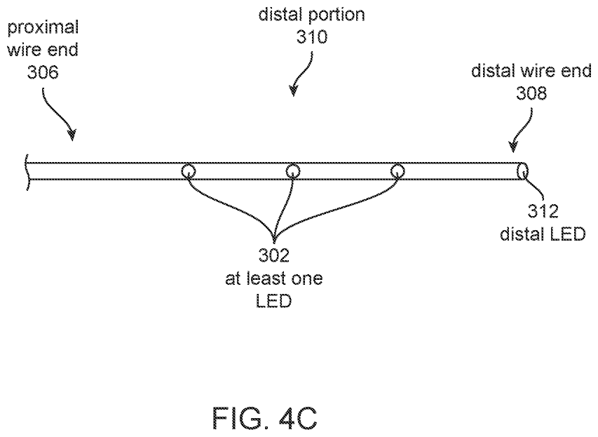

FIG. 4A illustrates a profile view of a catheter wire 30 including LEDs 302 throughout. The catheter wire 30 includes a proximal wire end 306 and a distal wire end 308 opposite the proximal wire end 306. It is also illustrated in FIG. 4A that the wire 30 includes three examples of locations for the at least one LED 302a, 302b, and 302c. While three LEDs (302a, 302b, and 302c) are illustrated in FIG. 4A, this is not intended to be limiting, and it is understood that as few as one LED 302 and as many LEDs 302 as may fit within the body of the wire 30 may be located within the wire 30 and at any place along the wire 30. As FIG. 4A shows the wire 30 as being straight, the wire 30 may be physically invisible extracorporeally, while the at least one LED 302 may be visible extracorporeally. The wire 30 may include a diameter that is consistent, varying, growing, decreasing, and combinations thereof throughout its length. The at least one LED 302 may include a size and luminosity that is consistent, varying, growing, decreasing, and combinations thereof.

FIG. 4B illustrates a profile view of a catheter wire 30, including at least a distal LED 316. The catheter wire 30 includes a proximal wire end 306 and a distal wire end 308 opposite the proximal wire end 306. It is also illustrated in FIG. 4B that the wire 30 includes one example for the location of the at least one LED 302 on the distal wire end 308 and no other LEDs 302 in the distal portion 310. However, this is not intended to be limiting, and it is understood that as few as one LED 302 and as many LEDs 302 as may fit within the body of the wire 30 may be located within the wire 30 and at any place along the wire 30. As FIG. 4B shows the wire 30 as being straight, the wire 30 may be physically invisible extracorporeally, while the at least one LED 302 may be visible extracorporeally. The wire 30 may include a diameter that is consistent, varying, growing, decreasing, and combinations thereof throughout its length. The at least one LED 302 may include a size and luminosity that is consistent, varying, growing, decreasing, and combinations thereof.

FIG. 4C illustrates a profile view of a catheter wire 30 including combinations of LEDs 302 as presented in FIGS. 4A and 4B. The catheter wire 30 includes a proximal wire end 306 and a distal wire end 308 opposite the proximal wire end 306. It is also illustrated in FIG. 4C that the wire 30 includes three examples of locations for the at least one LED 302. While three LEDs 302 are illustrated in FIG. 4C, this is not intended to be limiting, and it is understood that as few as one LED 302 and as many LEDs 302 as may fit within the body of the wire 30 may be located within the wire 30 and at any place along the wire 30. As FIG. 4C shows the wire 30 as being straight, the wire 30 may be physically invisible extracorporeally, while the at least one LED 302 may be visible extracorporeally. The wire 30 may include a diameter that is consistent, varying, growing, decreasing, and combinations thereof throughout its length. The at least one LED 302 may include a size and luminosity that is consistent, varying, growing, decreasing, and combinations thereof.

As previously iterated, throughout the specification, the wire 30 is disclosed as possibly comprising at least one LED 302. However, it is understood that the present specification is not solely limited to LEDs 302 located within a wire 30. The present specification also enables the use of a hypotube, a catheter shaft, combinations thereof, and in combination with a wire 30 for the location of the at least one LED 302. Additionally, the at least one LED 302 may be in or on the surface of any of these components.

Furthermore, while FIGS. 3A, 3B and 3C illustrate sinusoidal distal portions of the wire, and FIGS. 4A, 4B, and 4C illustrate straight distal portions of the wire, the present disclosure may be combined with any endovascular device. This includes, but is not limited to, endovascular devices that include a straight catheter, a sinusoidal distal end, a deflected tip or distal portion, an urged tip or distal portion, a flowered distal end, a hockey stick-shaped distal end, and a pigtailed distal end.

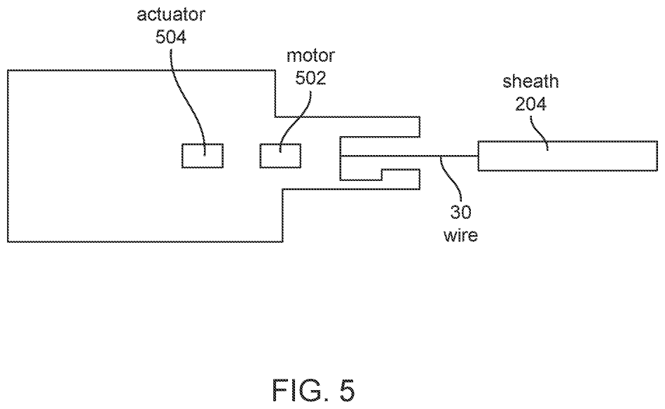

FIG. 5 illustrates a diagrammatic, side, cross-sectional view of a medical device 10. The medical device 10 may include a motor 502 to provide output. This output may include horizontal, vertical, rotational, other types of movement, or a combination thereof. The medical device 10 may include an actuator 504 to activate, deactivate, or combinations thereof, the motor 502. FIG. 5 additionally illustrates a sheath 204 around the wire 30, beginning partially down the length of the wire 30. However, the sheath 204 may start at any portion of the wire 30, including at the beginning of the wire 30. Throughout the specification, the catheter 40 is disclosed as comprising a wire 30. However, it is understood that the present specification is not limited to the use of a wire 30. The present specification also enables the use of a hypotube, a catheter shaft, combinations thereof, and in combination with a wire 30. FIG. 5 also shows one possible shape of the medical device 10, although the shape of the medical device 10 is not limited to a shape matching the representation in FIG. 5.

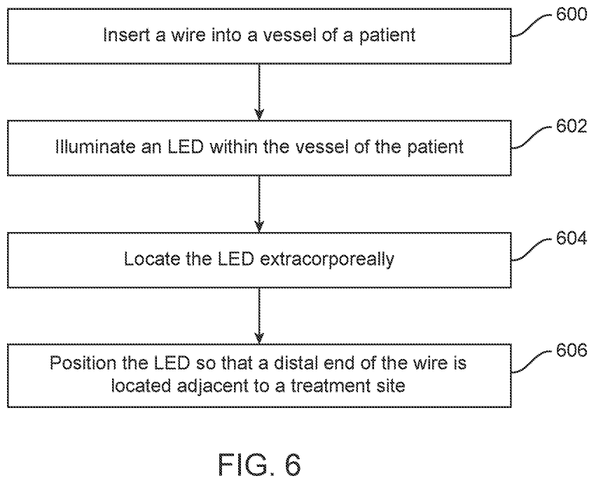

FIG. 6 illustrates a flow chart depicting a method of locating a wire, according to some examples. In some examples, the method of locating a wire includes inserting a wire into a vessel of a patient (at step 600). This wire may be at least partially contained within a sheath or other such tubular structure. According to some examples, the method of locating a wire includes illuminating an LED within the vessel of the patient (at step 602). This LED may be located at any portion of the wire, including at the distal end.

The method of locating a wire may include locating the LED extracorporeally (at step 604). One possible benefit of this method is that the light from the LED may be visible from outside of the patient, and the wire may be located without using expensive and sometimes cost-prohibitive imaging devices, such as ultrasound or radiograph devices. In some examples, the method of locating a wire includes positioning the LED so that a distal end of the wire is located adjacent to a treatment site (at step 606). While an LED located at the distal end of the wire would be beneficial to this step, it is not required.

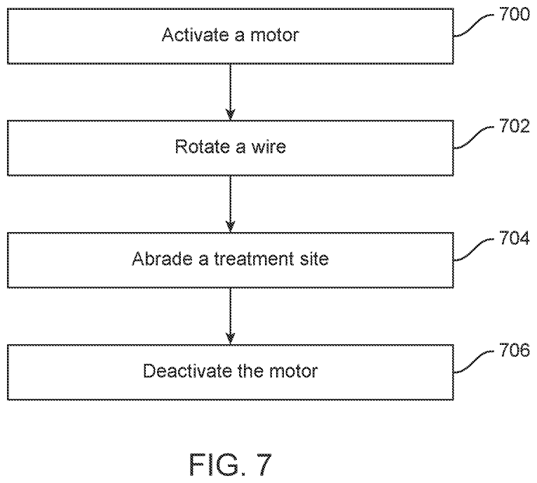

FIG. 7 illustrates a flow chart depicting a method of abrading a treatment site, according to some examples. In some examples, the method of abrading a treatment site includes activating a motor (at step 700). While the use of a motor to power the medical device is beneficial, other sources of power, including manual, may also provide the same benefit. The method of abrading a treatment site may include rotating a wire (at step 702). Rotating the wire may abrade the interior walls of a vessel.

According to some examples, the method of abrading a treatment site includes abrading a treatment site (at step 704). While rotating the wire may be beneficial to abrading the treatment site, such as if the wire includes a shape that may make contact with the sides of a vessel, other movements of the wire may also be used to cause abrasion. In some examples, the method of abrading a treatment site includes deactivating the motor (at step 706). If a different power source than the motor is used, this step may include deactivating that other power source instead of or in addition to the motor. Deactivating the motor allows the medical device to be safely retrieved or, in the case of multiple treatment sites, safely moved to a subsequent treatment site without the risk of unintentional abrasion of a non-treatment area.

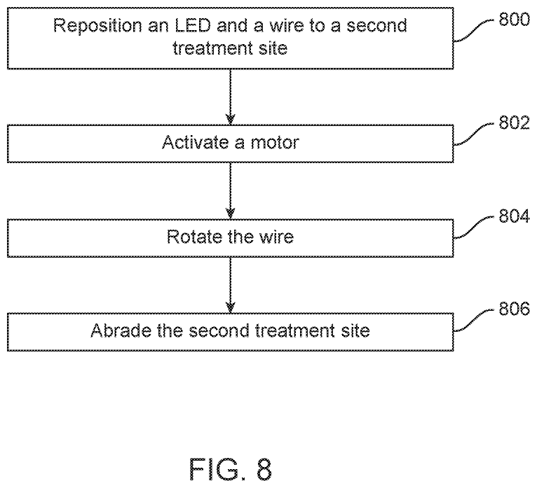

FIG. 8 illustrates a flow chart depicting a method of abrading a treatment site, according to some examples. In some examples, the method of abrading a treatment site includes repositioning an LED and a wire to a second treatment site (at step 800). This may be beneficial in instances where there are multiple treatment sites intended to be abraded or when the first treatment site is found to be unable to be abraded or should not be abraded. Additionally, the second treatment site may be a treatment site other than the second site in that treatment. For example, the second treatment site may be the second site relative to a previous treatment site, the previous treatment site being any treatment site other than the first treatment site.

According to some examples, the method of abrading a treatment site includes activating a motor (at step 802). While the use of a motor to power the medical device is beneficial, other sources of power, including manual, may also provide the same benefit. The method of abrading a treatment site may include rotating the wire (at step 804). Rotating the wire may abrade a vessel. According to some examples, the method of abrading a treatment site includes abrading the second treatment site (at step 806). While rotating the wire may be beneficial to abrading the second treatment site, such as if the wire includes a shape that can make contact with the sides of a vessel, other movements of the wire may also be used to cause abrasion.

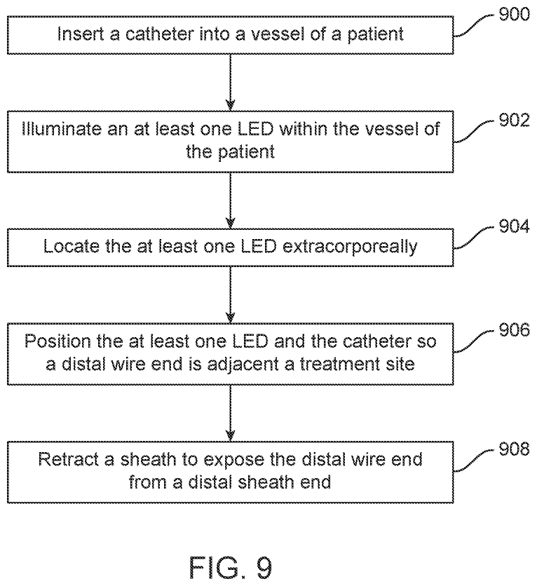

FIG. 9 illustrates a flow chart depicting a method of locating at least one LED, according to some examples. In some examples, the method of locating an at least one LED includes inserting a catheter into a vessel of a patient (at step 900). A wire or other type of tubular structure (e.g., a hypotube) may alternatively be used for this.

According to some examples, the method of locating an at least one LED includes illuminating an at least one LED within the vessel of the patient (at step 902). The illumination may be achieved by means of providing power to the LED. The method of locating an at least one LED may include locating the at least one LED extracorporeally (at step 904). This may be done visually or through other means. For example, the at least one LED may be located extracorporeally through tactile means.

In some examples, the method of locating an at least one LED includes positioning the at least one LED and the catheter, so a distal wire end is adjacent to a treatment site (at step 906). Positioning the at least one LED and the catheter at this location may be beneficial to ensure that the correct location is abraded. According to some examples, the method of locating an at least one LED includes retracting a sheath to expose the distal wire end from a distal sheath end (at step 908). In the case previously mentioned where only a wire is used, steps 906 and 908 may not require the catheter to be moved and the sheath to be retracted.

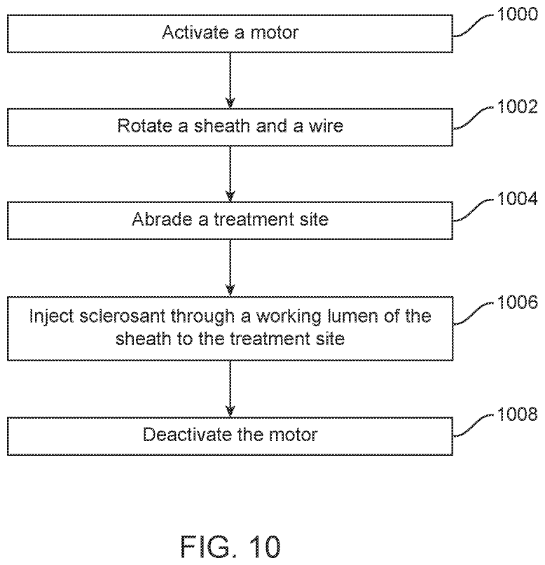

FIG. 10 illustrates a flow chart depicting a method of abrading a treatment site, according to some examples. In some examples, the method of abrading a treatment site includes activating a motor (at step 1000). While the use of a motor to power the medical device is beneficial, other sources of power, including manual, may also provide the same benefit. According to some examples, the method of abrading a treatment site includes rotating a sheath and a wire (at step 1002). Rotating the sheath and the wire may abrade a vessel.

The method of abrading a treatment site may include abrading a treatment site (at step 1004). While rotating the wire may be beneficial to abrading the second treatment site, such as if the wire includes a shape that can make contact with the sides of a vessel, other movements of the wire may also be used to cause abrasion. The wire may be located within the sheath, in another tubular structure, without a tubular structure or sheath, or any combination of these. The wire may be replaced by the sheath, another tubular structure, or any combination of these.

In some examples, the method of abrading a treatment site includes injecting sclerosant through a working lumen of the sheath to the treatment site (at step 1006). The sclerosant may alternatively be injected through any other means at the treatment site instead of or in addition to through the working lumen of the sheath. According to some examples, the method of abrading a treatment site includes deactivating the motor (at step 1008). If a different power source than the motor is used, this step may include deactivating that other power source instead of or in addition to the motor. Deactivating the motor allows the medical device to be safely retrieved or, in the case of multiple treatment sites, safely moved to a subsequent treatment site without the risk of unintentional abrasion of a non-treatment area.

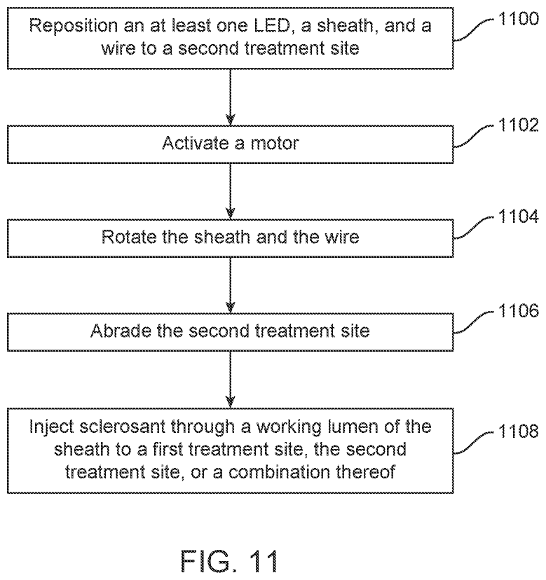

FIG. 11 illustrates a flow chart depicting a method of abrading a treatment site, according to some examples. In some examples, the method of abrading a treatment site includes repositioning an at least one LED, a sheath, and a wire to a second treatment site (at step 1100). This may be beneficial when there are multiple treatment sites intended to be abraded or when the first treatment site is found unable to be abraded or should not be abraded. Additionally, the second treatment site may be a treatment site other than the second site in that treatment. For example, the second treatment site may be the second site relative to a previous treatment site, the previous treatment site being a treatment site other than the first treatment site. The wire may be within the sheath, in another tubular structure, without a tubular structure or sheath, or any combination of these. The sheath, another tubular structure, or any combination of these may replace the wire.

According to some examples, the method of abrading a treatment site includes activating a motor (at step 1102). While the use of a motor to power the medical device is beneficial, other sources of power, including manual, may also provide the same benefit. The method of abrading a treatment site may include rotating the sheath and the wire (at step 1104). Rotating the wire may abrade the interior walls of a vessel.

In some examples, the method of abrading a treatment site includes abrading the second treatment site (at step 1106). While rotating the wire may be beneficial to abrading the second treatment site, such as if the wire includes a shape that can make contact with the sides of a vessel, other movements of the wire may also be used to cause abrasion. According to some examples, the method of abrading a treatment site may include injecting sclerosant through a working lumen of the sheath to a first treatment site, the second treatment site, or a combination thereof (at step 1108). The first treatment site may be a treatment site other than the first site in that treatment. For example, the first treatment site may be the first site relative to a following site, the following site not being the second treatment site.

The present disclosure includes a method of locating a treatment site of a patient and treating a vascular disease, comprising inserting a wire into a vessel of the patient. In some examples, the wire has a distal end and a proximal end located opposite the distal end. According to some examples, the wire is coupled to a control device at the proximal end. The method of locating a treatment site of a patient and treating a vascular disease may comprise an at least one light-emitting diode (LED) disposed in a distal portion of the wire. In some examples, the distal portion is adjacent the distal end. According to some examples, the method of locating a treatment site of a patient and treating a vascular disease comprises illuminating, via the control device, the at least one LED within the vessel of the patient. The method of locating a treatment site of a patient and treating a vascular disease may comprise locating, extracorporeally, the at least one LED. In some examples, the method of locating a treatment site of a patient and treating a vascular disease comprises positioning, via the at least one LED, the wire such that the distal end is located adjacent the treatment site.

According to some examples, the method of locating a treatment site of a patient and treating a vascular disease further comprises the control device that includes a motor configured to provide rotational output. An actuator may be electronically coupled to the motor. In some examples, the actuator is configured to activate and deactivate the motor. According to some examples, the method of locating a treatment site of a patient and treating a vascular disease further comprises activating the motor and rotating the wire in response to activating the motor. The wire may comprise an at least partially sinusoidal shape. In some examples, the method of locating a treatment site of a patient and treating a vascular disease further comprises abrading the treatment site in response to rotating the wire. According to some examples, the method of locating a treatment site of a patient and treating a vascular disease further comprises deactivating the motor.

In some examples, the treatment site is a first treatment site. According to some examples, the method of locating a treatment site of a patient and treating a vascular disease further comprises repositioning, via the at least one LED, the wire to a second treatment site. The method of locating a treatment site of a patient and treating a vascular disease may further comprise activating the motor and rotating the wire in response to activating the motor. In some examples, the method of locating a treatment site of a patient and treating a vascular disease further comprises abrading the second treatment site in response to rotating the wire.

The present disclosure also includes a method of locating a treatment site of a patient and providing a sclerotherapy procedure comprising inserting a catheter into a vessel of a patient. In some examples, the catheter includes a sheath. According to some examples, the sheath has a working lumen, a proximal sheath end, and a distal sheath end opposite the proximal sheath end. The proximal sheath end may be coupled to a control device. In some examples, the distal sheath end is configured for insertion into a vessel of a patient. According to some examples, the catheter includes a wire. The wire may extend from the control device through the working lumen of the sheath to the distal sheath end. In some examples, the wire has a proximal wire end and a distal wire end opposite the proximal wire end.

According to some examples, an at least one light-emitting diode (LED) is disposed in the distal wire end. The method of locating a treatment site of a patient and providing a sclerotherapy procedure may comprise illuminating, via the control device, the at least one LED within the vessel of the patient. In some examples, the method of locating a treatment site of a patient and providing a sclerotherapy procedure comprises locating, extracorporeally, the at least one LED. According to some examples, the method of locating a treatment site of a patient and providing a sclerotherapy procedure comprises positioning, via the at least one LED, the catheter such that the distal wire end is located adjacent to the treatment site. The method of locating a treatment site of a patient and providing a sclerotherapy procedure may comprise retracting the sheath to expose the distal wire end from the distal sheath end.

In some examples, the control device further includes a motor. According to some examples, the motor is configured to provide rotational output. The distal sheath end may be coupled to the motor. In some examples, an actuator is electronically coupled to the motor. According to some examples, the actuator is configured to activate and deactivate the motor. The method of locating a treatment site of a patient and providing a sclerotherapy procedure may further comprise activating the motor. In some examples, the method of locating a treatment site of a patient and providing a sclerotherapy procedure further comprises rotating the sheath and the wire in response to activating the motor. According to some examples, the wire comprises an at least partially sinusoidal shape. The method of locating a treatment site of a patient and providing a sclerotherapy procedure may further comprise abrading the treatment site in response to rotating the sheath and the wire.

In some examples, the method of locating a treatment site of a patient and providing a sclerotherapy procedure further comprises injecting sclerosant through the working lumen of the sheath to the treatment site. According to some examples, the method of locating a treatment site of a patient and providing a sclerotherapy procedure further comprises deactivating the motor. The treatment site may be a first treatment site. In some examples, the method of locating a treatment site of a patient and providing a sclerotherapy procedure further comprises repositioning, via the at least one LED, the sheath, and the wire to a second treatment site. According to some examples, the method of locating a treatment site of a patient and providing a sclerotherapy procedure further comprises activating the motor. The method of locating a patient's treatment site and providing a sclerotherapy procedure may include rotating the sheath and the wire in response to activating the motor. In some examples, the method of locating a treatment site of a patient and providing a sclerotherapy procedure further comprises abrading the second treatment site in response to rotating the sheath and the wire. According to some examples, the method of locating a treatment site of a patient and providing a sclerotherapy procedure further comprises injecting sclerosant through the working lumen of the sheath to a treatment site. The treatment site may be selected from the group consisting of the first treatment site, the second treatment site, and combinations thereof.

The disclosure also includes a medical device for locating a treatment site, comprising a wire having a distal end and a proximal end located opposite the distal end. In some examples, the wire is coupled to a control device at the proximal end. According to some examples, the medical device comprises at least one light-emitting diode (LED) disposed in a distal portion of the wire. The distal portion may be adjacent to the distal end. In some examples, the at least one LED is configured to radially illuminate a treatment site such that the treatment site is visible extracorporeally. According to some examples, the control device is configured to provide power to the at least one LED.

The LEDs may be powered through the action of unsheathing the distal end of the wire or catheter. I.e., in some examples, a limit switch may be provided. The limit switch may prevent or permit power to the LED. According to some examples, this limit switch only provides power to the LED when the sheath is in a partial, or fully retracted position. Contra, in some examples, the limit switch prevents power from flowing to the LED when the sheath is in a partial or fully extended position. This is only one example of how a limit switch may be implemented in order to provide or prevent power from flowing to the LED(s), and any implementation of a limit switch, or equivalent method of providing and preventing power from flowing with respect to the position of a sheath about the wire or catheter may be used.

In addition, the LEDs may be powered separately and independently from any other component of the system, including other LEDs. For example, an operator may want to power individual LEDs, or power the LEDs on an individual basis. In such examples, the system may include an actuator for providing and preventing power to the LEDs. In other examples, multiple actuators may be provided for powering independent LEDs in systems with more than one LED. While the example of an actuator appears here, it is understood that other, equivalent methods of preventing and providing power to an LED, such as via a lever, switch, knob, dial, etc. may be used with the present disclosure.

In some examples, the medical device further comprises a distal LED located at the distal end of the wire. According to some examples, a plurality of LEDs of the at least one LED is located throughout the wire at various positions of the distal portion. The wire may comprise an at least partially sinusoidal shape. In some examples, a shape of the wire is configured to be visible extracorporeally. According to some examples, a plurality of LEDs of the at least one LED is located along the at least partially sinusoidal shape.

The present disclosure also includes a sclerotherapy device comprising a catheter. The catheter may include a sheath. In some examples, a sheath has a working lumen, a proximal sheath end, and a distal sheath end opposite the proximal sheath end. According to some examples, the proximal sheath end is coupled to a control device. The control device may include a motor and an actuator. In some examples, the distal sheath end is configured for insertion into a vessel of a patient. The catheter may include a wire. According to some examples, the wire extends from the control device through the working lumen of the sheath to the distal sheath end. The wire may have a proximal wire end and a distal wire end opposite the proximal wire end. According to some examples, the catheter includes an at least one light- emitting diode (LED). In some examples, the at least one light-emitting diode (LED) is disposed in a distal portion of the wire. According to some examples, the distal portion is adjacent to the distal wire end.

In some examples, the distal sheath end is open. According to some examples, the catheter is configured to deliver sclerosant through the working lumen and out the distal sheath end to a treatment site. The motor may be configured to provide rotational output. In some examples, the motor is coupled to the proximal sheath end. According to some examples, the actuator is electronically coupled to the motor. The actuator may be configured to activate and deactivate the motor. In some examples, the sclerotherapy device further comprises a distal LED located at a distal wire end. According to some examples, a plurality of LEDs of the at least one LED is located throughout the wire at various positions of distal portion. The wire may comprise an at least partially sinusoidal shape. In some examples, a shape of the wire is configured to be visible extracorporeally. According to some examples, a plurality of LEDs of the at least one LED is located throughout the at least partially sinusoidal shape.

None of the steps described herein is essential or indispensable. Any of the steps can be adjusted or modified. Other or additional steps can be used. Any portion of any of the steps, processes, structures, and/or devices disclosed or illustrated in one embodiment, flowchart, or example in this specification can be combined or used with or instead of any other portion of any of the steps, processes, structures, and/or devices disclosed or illustrated in a different embodiment, flowchart, or example. The embodiments and examples provided herein are not intended to be discrete and separate from each other.

The section headings and subheadings provided herein are nonlimiting. The section headings and subheadings do not represent or limit the full scope of the embodiments described in the sections to which the headings and subheadings pertain. For example, a section titled “Topic 1” may include embodiments that do not pertain to Topic 1, and embodiments described in other sections may apply to and be combined with embodiments described within the “Topic 1” section.

To increase the clarity of various features, other features are not labeled in each figure.

The various features and processes described above may be used independently of one another or may be combined in various ways. All possible combinations and subcombinations are intended to fall within the scope of this disclosure. In addition, certain method, event, state, or process blocks may be omitted in some implementations. The methods, steps, and processes described herein are also not limited to any particular sequence, and the blocks, steps, or states relating thereto can be performed in other sequences that are appropriate. For example, described tasks or events may be performed in an order other than the order specifically disclosed. Multiple steps may be combined in a single block or state. The example tasks or events may be performed in serial, parallel, or some other manner. Tasks or events may be added to or removed from the disclosed example embodiments. The example systems and components described herein may be configured differently than described. For example, elements may be added to, removed from, or rearranged compared to the disclosed example embodiments.

Conditional language used herein, such as, among others, “can,” “could,” “might,” “may” “e.g.,” and the like, unless expressly stated otherwise, or otherwise understood within the context as used, is generally intended to convey that certain embodiments include, while other embodiments do not include, certain features, elements and/or steps. Thus, such conditional language is not generally intended to imply that features, elements, and/or steps are in any way required for one or more embodiments or that one or more embodiments necessarily include logic for deciding, with or without author input or prompting, whether these features, elements and/or steps are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y, and Z,” unless expressly stated otherwise, is otherwise understood with the context as used in general to convey that an item, term, etc. may be either X, Y, or Z. Thus, such conjunctive language is not generally intended to imply that certain embodiments require at least one of X, at least one of Y, and at least one of Z to each be present.

The term “and/or” means that “and” applies to some embodiments and “or” applies to some embodiments. Thus, A, B, and/or C can be replaced with A, B, and C written in one sentence and A, B, or C written in another sentence. A, B, and/or C means that some embodiments can include A and B, some embodiments can include A and C, some embodiments can include B and C, some embodiments can only include A, some embodiments can include only B, some embodiments can include only C, and some embodiments can include A, B, and C. The term “and/or” is used to avoid unnecessary redundancy.

While certain example embodiments have been described, these embodiments have been presented by way of example only and are not intended to limit the scope of the inventions disclosed herein. Thus, nothing in the foregoing description implies that any particular feature, characteristic, step, module, or block is necessary or indispensable. Indeed, the novel methods and systems described herein may be embodied in a variety of other forms; furthermore, various omissions, substitutions, and changes in the form of the methods and systems described herein may be made without departing from the spirit of the inventions disclosed herein.

We claim:

1. A method of locating a treatment site of a patient, comprising:
inserting a wire into a vessel of the patient, the wire having a distal end and a proximal end located opposite the distal end, the wire coupled to a control device at the proximal end, wherein at least one light-emitting diode (LED) is disposed in a distal portion of the wire, the distal portion adjacent the distal end;
illuminating, via the control device, the at least one LED within the vessel of the patient;
locating, extracorporeally, the at least one LED; and
positioning, via the at least one LED, the wire such that the distal end is located adjacent to the treatment site.

2. The method of claim 1, wherein the control device includes a motor configured to provide rotational output and an actuator electronically coupled to the motor whereby the actuator is configured to activate and deactivate the motor, the method further comprising activating the motor and rotating the wire in response to activating the motor.

3. The method of claim 2, wherein the wire comprises an at least partially sinusoidal shape, the method further comprising abrading the treatment site in response to rotating the wire.

4. The method of claim 3, further comprising deactivating the motor.

5. The method of claim 4, wherein the treatment site is a first treatment site, the method further comprising:
repositioning, via the at least one LED, the wire to a second treatment site;
activating the motor and rotating the wire in response to activating the motor; and
abrading the second treatment site in response to rotating the wire.

6. A method of locating a treatment site of a patient, comprising:
inserting a catheter into a vessel of a patient, the catheter including:
a sheath having a working lumen, a proximal sheath end, and a distal sheath end opposite the proximal sheath end, wherein the proximal sheath end is coupled to a control device, and the distal sheath end is configured for insertion into a vessel of a patient; and
a wire extending from the control device through the working lumen of the sheath to the distal sheath end, the wire having a proximal wire end and a distal wire end opposite the proximal wire end, wherein at least one light-emitting diode (LED) is disposed in the distal wire end;
illuminating, via the control device, the at least one LED within the vessel of the patient;
locating, extracorporeally, the at least one LED, and positioning, via the at least one LED, the catheter such that the distal wire end is located adjacent to the treatment site; and
retracting the sheath to expose the distal wire end from the distal sheath end.

7. The method of claim 6, wherein the control device further includes a motor configured to provide rotational output, wherein the distal sheath end is coupled to the motor, and an actuator electronically coupled to the motor whereby the actuator is configured to activate and deactivate the motor, the method further comprising activating the motor, and rotating the sheath and the wire in response to activating the motor.

8. The method of claim 7, wherein the wire comprises an at least partially sinusoidal shape, the method further comprising abrading the treatment site in response to rotating the sheath and the wire.

9. The method of claim 8, further comprising injecting sclerosant through the working lumen of the sheath to the treatment site.

10. The method of claim 8, further comprising deactivating the motor.

11. The method of claim 10, wherein the treatment site is a first treatment site, the method further comprising:
repositioning, via the at least one LED, the sheath and the wire to a second treatment site;
activating the motor and rotating the sheath and the wire in response to activating the motor; and
abrading the second treatment site in response to rotating the sheath and the wire.

12. The method of claim 11, further comprising injecting sclerosant through the working lumen of the sheath to a treatment site selected from the group consisting of the first treatment site, the second treatment site, and combinations thereof.

13. A medical device for locating a treatment site, comprising:
a wire having a distal wire end and a proximal wire end located opposite the distal wire end, the wire coupled to a control device at the proximal wire end; and
at least one light-emitting diode (LED) disposed in a distal portion of the wire, the distal portion adjacent to the distal wire end, the at least one LED configured to radially illuminate a treatment site such that the treatment site is visible extracorporeally, wherein the control device is configured to provide power to the at least one LED.

14. The medical device of claim 13, further comprising a distal LED located at the distal wire end.

15. The medical device of claim 13, wherein a plurality of LEDs of the at least one LED is located throughout the wire at various positions of the distal portion.

16. The medical device of claim 13, wherein the wire comprises an at least partially sinusoidal shape.

17. The medical device of claim 16, wherein a shape of the wire is configured to be visible extracorporeally.

18. The medical device of claim 17, wherein a plurality of LEDs of the at least one LED is located along the at least partially sinusoidal shape.

19. The medical device of claim 13, wherein the control device comprises a motor and an actuator, and the motor is coupled to the proximal wire end, and
wherein the actuator is electronically coupled to the motor, and the actuator is configured to activate and deactivate the motor.

20. The medical device of claim 13, wherein a shape of the wire is configured to be visible extracorporeally.
